(19) United States
(12) Patent Application Publication
(10) Pub. No.: US 2010/0004503 A1
(43) Pub. Date: Jan. 7, 2010

(54) APPARATUS AND A METHOD OF ENHANCING A PERSON'S SPORTING ABILITY

(75) Inventor: Leslie Foster, Bur Dubai (AE)

Correspondence Address: ABELMAN, FRAYNE & SCHWAB, 666 THIRD AVENUE, 10TH FLOOR, NEW YORK, NY, 10017, US

(73) Assignee: HYPNOSURF PTY LTD, Southport, QLD (AU)

(21) Appl. No.: 12/448,402

(22) PCT Filed: Dec. 18, 2007
(86) PCT No.: PCT/AU2007/001964

§ 371 Date: Jun. 17, 2009

(30) Foreign Application Priority Data
Dec. 18, 2006 (AU) 2006907057

(51) Int. Cl. A61M 21/00

(52) U.S. Cl. 600/26

(57) ABSTRACT

This invention relates a recording that is adapted to enhance a person's ability to perform one or more surfing related tasks. The recording includes a hypnotic stage that includes words and/or phrases and/or sentences and/or sounds that are adapted to induce a state of hypnosis in the person when the while the person is listening to the recording. The hypnotic stage of the recording is followed by one or more behavioural messages or instructions that are directed at surfing related actions the person must execute, namely to “paddle hard”, “charge in” and “stay focused”, if the surfing related task or tasks are to be performed or performed with a predetermined level of expertise, and wherein each of said behavioural messages or instructions is subsequently reinforced at least once during the recording by at least one affirmation concerning the person's ability to execute said messages or instructions.

### FIELD OF THE INVENTION

This invention relates to both apparatus and a method of enhancing a person's ability to perform one or more sports related tasks.

This invention has particular, but not exclusive application to both apparatus and a method of enhancing a person's ability to ride waves while supported on craft that has been designed to ride said waves, such as a surfboard. However, it will be appreciated that the present invention has other applications including enhancing a person's ability to glide over snow while riding a snowboard or to traverse a pavement or road surface on a skateboard, hitherto referred to as “carveboard riding”.

### BACKGROUND ART

Most people who regularly engage in a sporting activity find that there are occasions when their performance of some sports related tasks is not as good as has been achieved in the past and that instead of feeling happy, they may feel disappointment about their performance. Having said this, there may be many reasons why persons do not perform sports related tasks as well as they may have on another occasion.

For example, such inconsistencies in performance may be due to a lack of concentration or focus on the part of the person concerned. This may be because the person is feeling stressed or is in a state of mind that is not conducive to them performing at their best.

It is also believed that a person's level of confidence may also effect their sporting performance and wherein it is noted that a lack of confidence is often the reason given for poor performances. It also believed that a person may lack confidence because they have never performed the sports related task before, or because they have not performed the sports related task recently, or their ability to perform the sports related task of late has been rather poor in comparison with past performances.

A lack of confidence may also the lead the person to have negative thoughts about themselves and/or their ability to perform sports related tasks. As a consequence the person may experience feelings of low self esteem and/or negative thoughts may inhibit their ability to focus on the task they are attempting to perform, thereby resulting in failure.

In order to overcome sports performance related problems caused by a lack of confidence; an incorrect state of mind; low self esteem and/or negative thoughts, many people have sought help from sports psychologists who use a variety of techniques, including affirmations and visualization, to alter the person's state of mind and/or their feelings.

People suffering from sports related performance problems have also been known to seek assistance from hypnotherapists, who attempt to solve psychological problems through affirmations, behavioural messages and visulaisation techniques that are delivered while the person is experiencing a state of hypnosis. The person will hopefully recall these affirmations and behavioural messages when the person attempts to perform the sports related task that hitherto has posed a problem to them.

Alternatively, there are also recordings that a person may listen to which shall induce in a person that is listening to same a state of hypnosis and wherein the recordings are designed to deliver affirmations and behaviour altering messages at predetermined intervals while the person is both conscious and subconscious.

However, while listening to the aforementioned recordings and/or the messages delivered by sports psychologists and hypnotherapists may be generally beneficial, in relation to sports such as surfing, snowboarding and carveboarding, it is believed that better sporting performances and/or an ability to perform at a higher level consistently may be achieved if the affirmations and/or behavioural messages relate specifically to one or more of these sports, all of which involve a board that a person rides while in either a standing or crouching position and wherein the person is required to execute similar moves. In particular, the inventor believes that a person's ability to perform tasks related to one or more of the aforementioned sports will be enhanced by developing patterns of behaviour the inventor has identified as being important through the use of affirmations and/or behavioural messages and/or visualization techniques that are delivered while the person is in both a conscious and/or a subconscious state.

It is therefore an object of the present invention to provide an apparatus and a method of enhancing a person's ability to perform one or more sports related tasks that will ameliorate at least some of the disadvantages of the prior art.

### DISCLOSURE OF THE INVENTION

With the foregoing in view, this invention in one aspect relates to a method of enhancing a person's ability to perform one or more sports related tasks, said method including:

inducing in the person a state of hypnosis;

suggesting to the person while they are in a state of hypnosis, one or more behavioural messages or instructions that must be followed if the sports related task or tasks are to be performed or performed with a predetermined level of expertise, and

supporting at least some of said behavioural messages or instructions with at least one affirmation concerning the person's ability to execute said messages or instructions.

A hypnotic state has been described as an altered state of consciousness with an associated heightened level of suggestibility.

Prior to inducing in the person a state of hypnosis, the method may include an introductory portion during which a detailed explanation about the sports related task or tasks is given, including how they ought to be performed, as well as the meaning of any terminology that will be used. During the introductory portion at least some of the behavioural messages or instructions may be delivered at least once.

In order to reach a state of hypnosis, the person must be relaxed. To assist the person to relax, the method may include a relaxation component that serves as a prelude to the person entering a state of hypnosis. The relaxation component may include the delivery of words and/or phrases and/or sentences and/or sounds that are designed to assist the person to relax.

Alternatively or in addition, the relaxation component may include a musical component. Preferably this musical component has an Alpha Rhythm, (8-12 Hz).

During the relaxation component, one or more of the behavioural messages or instructions may be delivered at least once. However it is preferred that during the relaxation component the behavioural messages or instructions are faintly repeated over and over again in the background.

In order to induce in the person a state of hypnosis, the method may include an induction component. During this component words and/or phrases and/or sentences and/or sounds may be delivered that are designed to assist with the transformation of the person's state of mind from a conscious to a subconscious state.

Alternatively or in addition, the induction component may include a musical component. Preferably this musical component has a Theta Rhythm, (3-7 Hz).

During the induction component, the behavioural messages or instructions are delivered, preferably more than once. For example, these behavioural messages or instructions may be faintly repeated over and over again in the background. It is also during this component that all or at least some of the affirmations are delivered.

The method may further include a visualization component that is delivered while the person is in a state of hypnosis but after the behavioural messages, instructions and affirmations have been delivered at least once, and wherein the visualization component may include words and/or phrases and/or sentences and/or sounds that will enable the subconscious person to visualize him or her performing the sports related task or tasks. During this visualization component at least some of the behavioural messages or instructions may be repeated and wherein these may be reinforced by one or more of the affirmations.

To assist the person to maintain their state of hypnosis during the visualization component, the visualization component may include a musical component. Preferably the musical component has a Theta Rhythm, (3-7 Hz).

The method may also include a complementary component that follows the visualization component. During the complementary component various affirmations that support the behavioural messages or instructions are delivered. These affirmations may be repeated more than once and wherein the person may be encouraged to also say said affirmations.

It is also believed that the person's ability to perform the sports related tasks would be enhanced if the person were, in a conscious state, to repeat to themselves the affirmations on a regular basis.

In one embodiment, the person may be guided through the various steps of the method by a second person, such as a psychologist or a hypnotherapist.

Alternatively the person may be guided through the various steps of the method by listening to a recording that shall include means for inducing in the person a state of hypnosis followed by the behavioural messages or instructions which are subsequently supported by one or more affirmations. The recording may also include the introductory portion referred to above.

Regardless of the means by which the method is delivered, be it with the assistance of another person or by listening to the recording, in order to achieve the object or objects of the invention, it is preferred that the person be subjected to the method more than once and preferably on a regular basis. Further, in order that the person may maintain their enhanced ability to perform the sports related tasks, it is believed that they must continue to subject themselves to the method, but wherein the elapsed time between applications of the method may be greater than that when the person was attempting to master the sports related tasks.

By way of example, in the case of a recording, it is recommended that the person listen to the recording for a minimum of thirty consecutive days, after which the person may elect to listen to the recording on a two days on two days off basis. In addition, the person may elect to skip listening to any introductory portion.

In another aspect, this invention relates to a recording that is adapted to enhance a person's ability to perform one or more sports related tasks, said recording including:

words and/or phrases and/or sentences and/or sounds that are adapted to induce a state of hypnosis in a person when listening to same,

followed by one or more behavioural messages or instructions that must be followed if the sports related task or tasks are to be performed or performed with a predetermined level of expertise, and

wherein each of said behavioural messages or instructions is subsequently reinforced at least once during the recording by at least one affirmation concerning the person's ability to execute said messages or instructions.

Preferably the behavioural messages or instructions are repeated more than once during the recording.

The recording may also include an introductory portion during which a detailed explanation about the sports related task or tasks is given, including how they ought to be performed, as well as the meaning of any terminology that will be used. During the introductory portion at least some of the behavioural messages or instructions may be delivered at least once.

In order to reach a state of hypnosis, the person must be relaxed. To assist the person to relax, the recording may include a relaxation component that serves as a prelude to the person entering a state of hypnosis. The relaxation component may include the delivery of words and/or phrases and/or sentences and/or sounds that are designed to assist the person to relax.

Alternatively or in addition, the relaxation component may include a musical component. Preferably this musical component has an Alpha Rhythm, (8-12 Hz).

During the relaxation component, one or more of the behavioural messages or instructions may be delivered at least once. However it is preferred that during the relaxation component the behavioural messages or instructions are faintly repeated over and over again in the background.

In order to induce in the person a state of hypnosis, the recording may include an induction component. During this component words and/or phrases and/or sentences and/or sounds may be delivered that are designed to assist with the transformation of the person's state of mind from a conscious to a subconscious state.

Alternatively or in addition, the induction component may include a musical component. Preferably this musical component has a Theta Rhythm, (3-7 Hz).

During the induction component, the behavioural messages or instructions are delivered, preferably more than once. For example, these behavioural messages or instructions may be faintly repeated over and over again in the background. It is also during this component that all or at least some of the affirmations are delivered.

The recording may also include a visualization component which is timed to be delivered after the behavioural messages, instructions and affirmations and wherein the visualization component may include words and/or phrases and/or sentences and/or sounds that will enable the subconscious listener to visualize him or her performing the sports related task or tasks. During this visualization component at least some of the behavioural messages or instructions may be repeated and wherein these may be reinforced by one or more of the affirmations.

To assist the person to maintain their state of hypnosis during the visualization component, the visualization component may include a musical component. Preferably the musical component has a Theta Rhythm, (3-7 Hz).

The recording may also include a complementary component that follows the visualization component. During the complementary component various affirmations that support the behavioural messages or instructions are delivered. These affirmations may be repeated more than once and wherein the person may be encouraged to also say said affirmations.

The recording may be a video or audio recording.

Where the sport relates to surfing, regardless of the person's skill level and the manner in which a person is guided through the various steps of the method, such as by listening to a recording or aided by a psychologist or a hypnotherapist, the behavioural messages or instructions are preferably directed at the following actions or tasks that the surfer must observe:
1. “paddle hard”
2. “charge in”
3. “stay focused”
   Accordingly, in another aspect, this invention relates to a recording that is adapted to enhance a person's ability to perform one or more surfing related tasks, said recording including:

words and/or phrases and/or sentences and/or sounds that are adapted to induce a state of hypnosis in a person when listening to same,

followed by one or more behavioural messages or instructions that are directed at surfing related actions the person must execute, namely to “paddle hard”, “charge in” and “stay focused”, if the surfing related task or tasks are to be performed or performed with a predetermined level of expertise, and

wherein each of said behavioural messages or instructions is subsequently reinforced at least once during the recording by at least one affirmation concerning the person's ability to execute said messages or instructions.

Further, the following terms or phrases may be used to covey these instructions:
1. “paddle hard”
2. “charge in”
3. “stay focused”

However, it will be appreciated that these are not the only words that may be used to convey these messages or instructions and that these words have been chosen only by way of example. For example, instead of saying “stay focused” the words, “mind on job” may be used.

The message or instruction “paddle hard” means the surfer when taking off on a wave must paddle their surfboard in an explosive type manner in order to push and break through the pressure plane of the water moving up the face of the wave to then let gravity take over pulling the board down the face of the wave.

The message or instruction “charge in” means the surfer when taking off on a wave must immediately choose their line and drive hard down that line with absolute determination and commitment to take control of the board on the wave.

The message or instruction “stay focused” means the surfer must keep their mind solely focused on the task of riding the wave and to set their sight and mind on where they wish to position themselves on the wave in order for their body and board to follow.

Where the person is a beginner, additional behavioural messages or instructions may also include some or all of the following terms and/or phrases:
1. “stance”;
2. “getting up”
3. “conserve your energy”
4. “do not panic”
5. “do not surf alone”
6. “do not surf isolated locations”
7. “oceans energy”
8. “surf survey”
9. “warm up”
10. “rips”
11. “sweep”
12. “lull”
13. “sets”
14. “paddle out time”
15. “paddling”
16. “out of the way”
17. “close out”
18. “jumping up”
19. “direction”
20. “awareness”
21. “sitting up”
22. “Eskimo roll”
23. “duck dive”
24. “catching the wave”

The term “stance” means the standing position the surfer must select, assume and use in the sport of surfing.

The term “getting up” means the surfer going from a lying down or kneeling position on the surfboard to a standing position.

The phrase “conserve your energy” means the beginner surfer needs to limit their energy use where possible.

The phrase “do not panic” means the beginner surfer needs to remain calm at all times.

The phrase “do not surf alone” means the beginner surfer must always have a buddy to surf with.

The phrase “do not surf in isolated locations” means the beginner surfer must never surf at isolated locations.

The term “oceans energy” means the beginner surfer needs to be aware of the power of the ocean and the energy of the waves at all times.

The term “surf survey” means the beginner surfer must study the surf conditions before entering the water.

The term “warm up” means the beginner surfer needs to warm the body before entering the water.

The term “rips” means the beginner surfer must always be aware of the under currents that can carry them out to sea and how they can be a surfers best friend when paddling out.

The term “sweep” means the beginner surfer must always be aware of the direction they may be traveling parallel to the shore unbeknown to them.

The term “lull” means when the quiet period of the incoming surf between the bigger sets of waves.

The term “sets” means the bigger waves coming through.

The term “paddle out time” means the beginner surfer needs to always paddle out during a lull in the surf.

The term “paddling” means when the technique and style the beginner surfer needs to select and use in the sport of surfing.

The term “out of the way” means the beginner surfer needs to stay out of the way of other surfers surfing the break and to never sit inside the breaking zone.

The term “close out” means the wave closes down and the complete face of the wave breaks simultaneously.

The term “jumping up” means the beginner surfer needs to get to their feet in a swift motion to a standing position to ride the wave.

The term “direction” means the beginner surfer must always be conscious of the direction they are paddling.

The term “awareness” means beginner surfer needs to be aware of the many aspects of mother nature before venturing into the water. They need to consider the type of surfboard, surf conditions, sun protection, safety aspects, sea creatures, other surfers and more.

The term “sitting up” means the surfer is in a sitting position on their surf board waiting for a wave or resting.

The term “Eskimo roll” means the surfer when paddling out into the surf in a laying down position rolls the board over 180 degrees to get through a breaking wave and immediately when the wave passes over they complete the roll over with a further 180 degree turn to complete the 360 degree rollover and to reposition themselves to resume paddling out.

The term “duck dive” means the surfer pushes the front of the board under a breaking wave by submerging the nose and then places weight with their foot on the back of the board to lift the nose of the board out of and to exit the back of the breaking wave. They simply duck dive the board under the breaking wave.

The term “catching the wave” means the surfer is paddling hard and is getting onto and catching then riding the wave.

Where the person's skill level is intermediate or advanced, additional behavioural messages or instructions may also include some or all of the following terms and/or phrases:
1. “take the drop”
2. “late drop”
3. “commit”
4. “bottom turn”
5. “lean in”
6. “find your line”
7. “racing down the line”
8. “trim the rail”
9. “carve”
10. “cut back”
11. “cutty”
12. “stall”
13. “drop knee”
14. “chest forward”
15. “off the lip”
16. “floater”
17. “hang ten”
18. “hang five”
19. “walk the board”
20. “clean exit”
21. “A” frame”
22. “right handers”
23. “left handers”
24. “barrel”
25. “tube”
26. “green room”
27. “filthy pit”
28. “tuck in”
29. “pull in”
30. “drag arm”
31. “rail grab”
32. “grab rail”
33. “grab the outside rail”
34. “air”
35. “down low”
36. “section”
37. “pitching”
38. “glides and runs”
39. “running”
40. “a surfers breath”
41. “boardies”
42. “shred”
43. “tear”
44. “line up”
45. “going hard”
46. “going off”
47. “going nuts”
48. “champ”
49. “sussed”
50. “face turn”

The term “take the drop” means the surfer takes the vertical drop down the face of the wave on take off. The surfer must “charge in” when taking the drop.

The term “late drop” means the surfer is executing a “take the drop” when the wave is about to break and the vertical drop down the face of the wave is at a very steep vertical angle. The surfer must “charge in” when taking the “late drop”.

The term “commit” means the surfer has committed to taking the wave. A surfer must commit with absolute determination and without hesitancy when taking off and riding the wave.

The term “bottom turn” means the surfer is making a turn at the bottom of the wave to position themselves on the wave face in order to surf further along the face of the wave.

The term “lean in” means the surfer has to lean in to turn the surfboard.

The term “find your line” means the surfer has to identify and position themselves on a correct line to achieve maximum power, trim, control and speed on the wave.

The term “racing down the line” means the surfer is moving at a really fast pace along the face of the wave in order to make the “section” and complete the ride.

The term “trim the rail” means the surfer positions the surfboard on the wave face with the correct area of surfboard rail (the side) and surfboard bottom in the wave face in order to have maximum control of the surfboard.

The term “carve” means the surfer carves the surfboard over many areas of the wave face in a controlled, flowing and composed manner.

The term “cut back” means when the surfer is riding along the face of the wave and then make a wide arcing or tight turn back into the power source of the wave.

The term “cutty” has the same meaning as “cut back”.

The term “stall” means the surfer intentionally slows the speed of the surf board by placing additional weight on the rear of the surf board to act like a type of brake.

The term “drop knee” means the surfer drops their knee nearest the rear of the board in order to execute numerous different types of surfing maneuvers.

The term “chest forward” means the surfer when riding the wave has their chest facing the front of the surfboard.

The term “off the lip” means the surfer takes the surf board from the bottom of the wave at high speed vertically up the face of the wave to the top of the wave then radically and quickly turns the surf board around to go back down the face of the wave.

The term “floater” means when the surfer is “racing down the line” and then turns the board to position themselves to ride across the top of the wave and as the wave is breaking they then turn the board down the front “section” of breaking white water heading towards or parallel to the shoreline and ride over the top of the white water.

The term “hang ten” means the surfer hangs ten toes over the front of the surfboard whilst riding the wave, often on a long board.

The term “hang five” means the surfer hangs five toes over the front of the surfboard whilst riding the wave, often on a long board.

The term “walk the board” means the surfer walks back and or forth along the length of the surfboard often with a cross step style on a longboard.

The term “clean exit” means the surfer completes riding the wave in a composed controlled manner.

The term “A” frame” means a wave is breaking in both a right hand and left hand direction. If looking from the shore line directly at the wave coming towards you it looks like an ‘A’ with the wave peeling off to both sides. Commonly referred to as the perfect wave if breaking in a clean sizeable manner.

The term “right handers” means a wave that is breaking in a right hand direction for the actual surfer paddling for that wave. So if looking from the shore line directly at the wave coming towards you it would be breaking from your right to your left.

The term “left handers” means a wave that is breaking in a left hand direction for the actual surfer paddling for that wave. So if looking from the shore line directly at the wave coming towards you it would be breaking from your left to your right.

The term “barrel” means the section of wave that forms the cylinder of air that is surrounded by water as the wave is curling over in preparation to fully break. The surfer would try to position themselves on the wave face inside the “barrel” to be completely surrounded by water. Often described as the ultimate surfing move and positioning on a wave.

The term “tube” has the same meaning as the “barrel”.

The term “green room” has the same meaning as the “barrel”.

The term “filthy pit” has the same meaning as “barrel” but on waves that are larger in size that are sucking and pitching out in a very dangerous life threatening manner. Experienced surfers only would pull in to a “filthy pit”.

The term “tuck in” means when the surfer has to adjust their body stance and positioning on the surfboard to fit inside or “tuck in” to the green room, tube or barrel.

The term “pull in” means when the surfer pulls into a green room, tube or barrel.

The term “drag arm” means when the surfer drags their inside arm in the water of the face of the wave to control their position and speed, often when inside or pulling into a barrel to control speed, positioning, angle of the board or other positioning moves.

The term “rail grab” means the surfer takes hold of a rail (the side) of the surfboard with a hand in order to obtain maximum stability and control to successfully execute numerous different types of surfing maneuvers. Often used when inside the green room, tube or barrel.

The term “grab rail” has the same meaning as “rail grab”.

The term “grab that outside rail” has the same meaning as “rail grab” but only the rail facing the shoreline or outside section of the breaking wave.

The term “air” means when the surfer and surfboard leave the face and water surface of the wave often with great speed and momentum and takes air in the process of executing radical maneuvers.

The term “down low” means when a surfer bends their knees and crouches down very low on their surfboard. Often to get inside a tight barrel or when riding through a white water section to gain greater stability.

The term “section” means a section of wave in front of the surfer that is walling up (forming a wall of water) or breaking. Often the surfer has to move quickly by finding the best line to beat or get past a section of the wave in order to continue riding further along the same wave.

The term “pitching” means when the wave is just about to break and the top of the wave is “pitching” out over the face of the wave.

The term “glides and runs” means when the surfer is paddling the surfboard with long deep strategically timed strokes allowing the surfboard to plane and skim over the water surface at a smooth fast pace, this is known as “glide and run” or “glides and runs” and often used for paddling distances as it will conserve energy in the surfer.

The term “running” has the same meaning as “glides and runs”. The surfer is paddling the surfboard in a manner that the surfboard is gliding and running over the surface of the water.

The term “a surfers breath” means the deep breath a surfer must hold at various times in the sport of surfing. A “surfers breath” is required by the surfer when being held under the water surface [held down].

The term “boardies” means the board shorts or trunks the surfer is wearing.

The term “shred” means the surfer is surfing radically making lots of tracks and shredding the water surface and the face of the wave to pieces.

The term “tear” has the same meaning as “shred”.

The term “line up” means the position the surfer will sit to catch a wave. The surfer will identify reference points (often landmarks) to position themselves in the “line up”.

The term “going hard” means when the surfer is giving it all they have, every bit of energy and every bit of effort, giving all they have.

The term “going off” means when the surfer is surfing exceptionally well at a very high standard.

The term “going nuts” has the same meaning as “going off”.

The term “champ” means the surfer is surfing like a champion.

The term “sussed” means when the surfer has a good understanding of the conditions and the way the surf is working in a particular surf session.

The term “face turn” means a turn the surfer makes a turn in the mid section of the face of the wave.

Where the sport relates to surfing, regardless of the person's skill level, the affirmations preferably relate to the following actions and/or attributes of the person, namely:
1. their ability to select the most appropriate wave to ride;
2. that they always paddle hard when attempting to catch a wave;
3. that they always charge in when catching a wave;
4. that they always stay focused when surfing a wave;
5. that their take offs are faultless, and
6. that they are a top surfer.

These affirmations may take the form of:
1. “My wave selection is always superb”;
2. “I always paddle hard when catching a wave”;
3. “I always charge in when catching a wave”;
4. “I always stay focused when surfing a wave”;
5. “My take offs are faultless”, and
6. “I am a good surfer”.

However, it will be appreciated that alternative words may be used to express the same affirmations and that these words have been chosen only by way of example.

Where the sport relates to snowboarding, the behavioural messages or instructions are preferably directed at the following actions or tasks that the surfer must observe:
1. “charge in”
2. “stay focused”

Further, the following terms or phrases may be used to covey these instructions:
1. “charge in”
2. “stay focused”

However, it will be appreciated that these are not the only words that may be used to convey these messages or instructions and that these words have been chosen only by way of example.

The message or instruction “charge in” means when the snowboarder is to commence their descent they must immediately choose their line and drive hard down that line with absolute determination and commitment to take control.

The message or instruction “stay focused” means the snowboarder must keep their mind solely focused on the task of snowboarding and set their sight and mind on where they wish to position themselves on the mountain, hill or slope in order for their body and board to follow.

Additional behavioural messages or instructions may also include some or all of the following terms and/or phrases:
1. “skidding”
2. “garland”
3. “grab”
4. “transition”
5. “halfpipe”
6. “quaterpipe”
7. “halfcab”
8. “heel side”
9. “hit”
10. “aerials”
11. “spins”
12. “lip”
13. “sideslip”
14. “toe side”
15. “traverse”
16. “wall”
17. “backside wall”
18. “frontside wall”
19. “freestyle”
20. “freeriding”
21. “flex”
22. “fall line”
23. “edge change”
24. “edge angle”
25. “downhill edge”
26. “catching air”
27. “angle of attack”
28. “shallow angle of attack”
29. “steep angle of attack”
30. “ground school”

The term “skidding” means the snowboarder is moving the board sideways as well as forward as a means to control speed.

The term “garland” means the snowboarder is making a series of partial turns without edge changes.

The term “grab” means the snowboarder grabs the tip, the tail, the toe or the heel edge of the board with their hand. Similar to surfing but the surfer grabs the rail of the board.

The term “transition” means the curve connecting the flat bottom of a ‘halfpipe’ and its vertical walls.

The term “halfpipe” means a U-shaped channel in the snow designed with smooth transition between the flat bottom and vertical sections allowing the snowboarder to go horizontal to vertical in a smooth transitional manner for acrobatic and aerial type maneuvers.

The term “quarterpipe” is half of a “halfpipe” and is a single snow wall with smooth transition between the flat bottom and vertical sections allowing the snowboarder to go horizontal to vertical in a smooth transitional manner for acrobatic and aerial type maneuvers.

The term “halfcab” means the snowboarder makes an advanced halfpipe move also called a jump-180.

The term “heel side” means the snowboarder makes a turn on the heel edge of the board.

The term “hit” means the snowboarder makes a jump.

The term “aerials” means maneuvers performed in the air.

The term “spins” means the snowboarder performs an aerial spin maneuver.

The term “lip” means the top edge of the wall in a half or quarter pipe.

The term “sideslip” refers to a maneuver which the snowboarder slides downhill with the board perpendicular to the fall line.

The term “toe side” refers to a turn made on the toe edge of the board.

The term “traverse” means the snowboarder takes the descent going diagonally back and forth across the slope.

The term “wall” means the side wall of a “halfpipe” and “quarterpipe”.

The term “backside wall” means the wall of the “halfpipe” your heel edge hits if you were riding forward.

The term “frontside wall” means the wall of the “halfpipe” closest to your toe edge.

The term “freestyle” means acrobatic snowboarding.

The term “freeriding” means all mountain riding.

The term “flex” means the amount of and position on the board flexing will occur.

The term “fall line” means the most direct path down the mountain, hill or slope.

The term “edge change” means the snowboarder moves smoothly from one edge of the board to the other to commence a new turn.

The term “edge angle” means the angle the snowboard is tilted on its edge.

The term “downhill edge” means the edge of the snowboard farthest down the mountain, hill or slope.

The term “catching air” has the same meaning as “air”.

The term “angle of attack” means the degree angle in which the snowboarder is descending down the mountain, hill or slope.

The term “shallow angle of attack” means the degree angle is mostly across the mountain, hill or slope providing a slower descent.

The term “steep angle of attack” means the degree angle is mostly downhill providing a fast descent.

The term “ground school” means practicing and mastering skills and maneuvers on flat ground.

Where the sport relates to snowboarding, the affirmations preferably relate to the following actions and/or attributes of the person, namely:
1. that they are a good snowboarder;
2. that they are a top snowboarder;
3. that they always charge in when commencing descent, and
4. that they always stay focused when making the descent.

These affirmations may take the form of:
1. I am a good snowboarder;
2. I am a top snowboarder;
3. I always charge in when commencing descent, and
4. I always stay focused when making the descent.

However, it will be appreciated that alternative words may be used to express the same affirmations and that these words have been chosen only by way of example.

Where the sport relates to carveboard riding, the behavioural messages or instructions are preferably directed at the following actions or tasks that the surfer must observe:

The term “rail grab” means the carveboarder takes hold of the edge of the carveboard with a hand in order to obtain maximum stability and control to successfully execute numerous different types of maneuvers.

The term “grab rail” has the same meaning as “rail grab”.

The phrase “grab that outside rail” has the same meaning as “rail grab” but only the rail facing the downhill or outside section of the ride.

The term “shred” means the carveboarder is riding radically back and forth over the asphalt surface.

The term “tear” has the same meaning as “shred”.

The term “carve” means the carveboarder is carving their way through the descent with a stylish technique and manner.

Where the sport relates to carveboarding, the affirmations preferably relate to the following actions and/or attributes of the person, namely:
1. that they are a good carveboarder;
2. that they are a top carveboarder;
3. that they always charge in when commencing descent, and
4. that they always stay focused when making the descent.

These affirmations may take the form of:
1. I am a good carveboarder;
2. I am a top carveboarder;
3. I always charge in when commencing descent, and
1. “charge in”
2. “stay focused”

Further, the following terms or phrases may be used to covey these instructions:
1. “charge in”
2. “stay focused”

However, it will be appreciated that these are not the only words that may be used to convey these messages or instructions and that these words have been chosen only by way of example.

The message or instruction “charge in” means when the carveboarder is to commence their descent they must immediately choose their line and drive hard down that line with absolute determination and commitment to take control.

The message or instruction “stay focused” means the carveboarder must keep their mind solely focused on the task of carveboarding and set their sight and mind on where they wish to position themselves on the hill or slope in order for their body and board to follow.

Additional behavioural messages or instructions may also include some or all of the following terms and/or phrases:
1. “rail grab”
2. “grab rail”
3. “grab that outside rail”
4. “shred”
5. “tear”
6. “carve”
4. I always stay focused when making the descent.

However, it will be appreciated that alternative words may be used to express the same affirmations and that these words have been chosen only by way of example.

In order that this invention may be more easily understood and put into practical application, below is an example of a script for a recording relating to surfboard riding that is directed to a person of intermediate/advanced skill level.

It is believed that if a person listens to the recording continuously for at least 30 days, and regularly thereafter, the person will notice an improvement in their ability to ride a surfboard and/or the quality of their performances will be consistently higher.

It will also be appreciated that listening to the recording will enable persons to maintain their skill levels during periods when they can not practice their skills by riding a surfboard, such as may be the case if they are injured.

It will also be appreciated that the above example is given as an illustration only of the present invention and that all such modifications and variations thereto as would be apparent to persons skilled in the art are deemed to fall within the broad scope and ambit of this invention as herein defined in the appended claims.

1. A recording that is adapted to enhance a person's ability to perform one or more surfing related tasks, said recording including:
words and/or phrases and/or sentences and/or sounds that are adapted to induce a state of hypnosis in a person when listening to same,
followed by one or more behavioural messages or instructions that are directed at surfing related actions the person must execute, namely to “paddle hard”, “charge in” and “stay focused”, if the surfing related task or tasks are to be performed or performed with a 10 predetermined level of expertise, and
wherein each of said behavioural messages or instructions is subsequently reinforced at least once during the recording by at least one affirmation concerning the person's ability to execute said messages or instructions.

2. A recording as claimed in claim 1, wherein the following terms are used to convey said behavioural messages or instructions:
1. “paddle hard”
2. “charge in”
3. “stay focused”

3. A recording as claimed in claim 2, wherein said behavioural message or instruction “paddle hard” means the surfer when taking off on a wave must paddle their surfboard in an explosive type manner in order to push and break through the pressure plane of the water moving up the face of the wave to then let gravity take over pulling the board down the face of the wave.

4. A recording as claimed in claim 2, wherein said behavioural message or instruction “charge in” means the surfer when taking off on a wave must immediately choose their line and drive hard down that line with absolute determination and commitment to take control of the board on the wave.

5. A recording as claimed in claim 2, wherein said behavioural message or instruction “stay focused” means the surfer must keep their mind solely focused on the task of riding the wave and to set their sight and mind on where they wish to position themselves on the wave in order for their body and board to follow.

6. A recording as claimed in claim 2, wherein said affirmations relate the following actions and/or attributes of the person, namely:
1. their ability to select the most appropriate wave to ride;
2. that they always paddle hard when attempting to catch a wave;
3. that they always charge in when catching a wave;
4. that they always stay focused when surfing a wave;
5. that their take offs are faultless, and
6. that they are a top surfer.

7. A recording as claimed in claim 6, wherein said affirmations are:
1. “My wave selection is always superb”;
2 “I always paddle hard when catching a wave”;
3. “I always charge in when catching a wave”;
4. “I always stay focused when surfing a wave”;
5. “My take offs are faultless”, and
6. “I am a good surfer”.

8. A recording as claimed in claim 2, wherein said recording includes a visualization component that is intended to be delivered while the person who is listening to said recording is in a state of hypnosis but after said behavioural messages, instructions and affirmations have been delivered, said visualization component including words and/or phrases and/or sentences and/or sounds that will enable the subconscious person to visualize him or her performing the surfing related tasks.

9. A recording as claimed in claim 8, wherein at least some of said behavioural messages or instructions are repeated during said visualization component and are reinforced by at least some of said affirmations.

10. A method of enhancing a person's ability to perform one or more surfing related tasks, said method including listening to the recording that is the subject of claim 1.

11. A recording that is adapted to enhance a person's ability to perform one or more sports related tasks, said recording including:
words and/or phrases and/or sentences and/or sounds that are adapted to induce a state of hypnosis in a person when listening to same,
followed by one or more behavioural messages or instructions that must be followed if the sports related task or tasks are to be performed or performed with a predetermined level of expertise, and
wherein each of said behavioural messages or instructions is subsequently reinforced at least once during the recording by at least one affirmation concerning the person's ability to execute said messages or instructions.

12. A method of enhancing a person's ability to perform one or more sports related tasks, said method including:
inducing in the person a state of hypnosis;
suggesting to the person while they are in a state of hypnosis, one or more behavioural messages or instructions that must be followed if the sports related ask or tasks are to be performed or performed with a predetermined level of expertise, and
supporting at least some of said behavioural messages or instructions with at least one affirmation concerning the person's ability to execute said messages or instructions.

13. A method of enhancing a person's ability to perform one or more sports related tasks as claimed in claim 12, wherein the sports related tasks relates to surfing and the behavioural messages or instructions are preferably directed at the following actions or tasks that the person must observe:
1. “paddle hard”;
2. “charge in”, and
3. “stay focused”.
